(19) United States
(12) Patent Application Publication
(10) Pub. No.: US 2025/0325622 A1
(43) Pub. Date: Oct. 23, 2025

(54) Autoimmune Disease Modulation Protocol

(71) Applicant: Keven A. M. Pimentel, Hallandale Beach, FL (US)

(72) Inventor: Keven A. M. Pimentel, Hallandale Beach, FL (US)

(21) Appl. No.: 18/638,361

(22) Filed: Apr. 17, 2024

(51) Int. Cl. A61K 36/81; A61K 31/05; A61K 31/122; A61K 31/145; A61K 31/194; A61K 31/197; A61K 31/593; A61K 33/08; A61K 45/06; A61P 29/00; A61P 37/02

(52) U.S. Cl. CPC A61K 36/81; A61K 31/05; A61K 31/122; A61K 31/145; A61K 31/194; A61K 31/197; A61K 31/593; A61K 33/08; A61K 45/06; A61P 29/00; A61P 37/02

(57) ABSTRACT

The invention presents a groundbreaking approach to safer autoimmune disease modulation, combining vitamins, minerals, and plant-based compounds for immune regulation, anti-inflammatory effects, and tissue regeneration. Key ingredients include Vitamin D3, Vitamin K2, a Magnesium Complex, dulcamara root, and Trans-Resveratrol, synergistically balanced for optimal efficacy. This protocol offers personalized dosing, adaptable to individual needs based on age, weight, and symptom severity. Available in various forms like capsules and tablets, it caters to diverse patient preferences. The invention not only alleviates symptoms but also targets the root causes of autoimmune imbalances, marking a significant advancement in autoimmune care.

### BACKGROUND OF THE INVENTION

Autoimmune diseases, characterized by the immune system mistakenly attacking healthy cells, affect millions of people globally. Current treatments, often immunosuppressive medications, may have limited effectiveness and adverse side effects. The invention proposes a novel, natural approach to treating autoimmune diseases, utilizing a synergistic combination of plant-derived compounds known for their therapeutic properties.

### FIELD OF INVENTION

The present invention relates to a supplement protocol for the modulation of autoimmune diseases & their symptoms, specifically leveraging the combined use of specific plant-based compounds and their synergistic affects on the body.

### BACKGROUND ART

The invention exhibits significant novelty and non-obviousness, particularly when juxtaposed with the current landscape of treatments for autoimmune diseases.

### Addressing Autoimmune Complications in Immunotherapy:

Current Immunotherapy Challenges: Immunotherapy, a significant advancement in treating certain diseases, unfortunately, has been linked to autoimmune complications. These complications can trigger or exacerbate autoimmune responses, posing a risk to patients with pre-existing autoimmune conditions.

Invention's Unique Approach: Unlike these treatments, the invention uses plant-based compounds, which are inherently less likely to provoke an autoimmune response. This approach could potentially offer a safer alternative for patients, particularly those who are at risk of immunotherapy-related autoimmune complications.

### Comprehensive Management of Autoimmune Diseases:

Limitations of Current Treatments: Traditional treatments often focus on symptom management, using medications like NSAIDs, corticosteroids, and immunosuppressants, which can have significant side effects and do not necessarily address the underlying immune dysregulation.

Holistic Approach of invention: In contrast, the invention aims to modulate the immune system and reduce inflammation through a blend of natural compounds. This strategy not only targets symptoms but also addresses the root causes of autoimmune responses, offering a more comprehensive management approach.

### Personalized and Adaptive Treatment Strategy:

One-Size-Fits-All Limitation: Many existing treatments do not adequately account for individual variability in disease manifestation and response to treatment. This can lead to less effective or even harmful outcomes for some patients.

Invention's Personalization: The protocol's adaptability to individual factors such as age, weight, and symptom severity represents a significant advancement. This personalized approach could lead to more effective and safer treatment outcomes for patients with autoimmune diseases.

### Safety and Minimization of Adverse Effects:

Adverse Effects of Current Medications: Current treatments, while effective for some, can lead to a range of adverse effects, from mild discomfort to severe complications, impacting patient quality of life.

Enhanced Safety Profile of invention: The invention, with its emphasis on natural, plant-based ingredients, aims to minimize these adverse effects. This focus on safety and tolerability is a novel aspect that distinguishes it from many existing autoimmune disease treatments.

The invention demonstrates clear novelty and non-obviousness along with a significant improvement upon current Art through its unique approach to managing autoimmune diseases. Its potential to mitigate immunotherapy-related complications, comprehensive immune modulation, personalized treatment strategy, and focus on safety and minimal adverse effects represent significant advancements over current treatment methodologies.

### BRIEF SUMMARY OF THE INVENTION

This invention introduces a novel plant-based supplement protocol specifically designed to address the challenges in modulating autoimmune diseases such as Rheumatoid arthritis (RA), Lupus, Multiple Sclerosis (MS), Psoriatic Arthritis, Graves disease, Hashimoto Thyroiditis, Antiphospholipid Syndrome (APS), Autoimmune Addison's Disease (AAD), Multiple Sclerosis, Sjögren's Syndrome, and Systemic Lupus Erythematosus (SLE). Unlike conventional treatments that often rely on immunosuppressive drugs with significant side effects, the invention offers a natural, holistic approach to treating auto immune diseases by aiding in immune system modulation, inflammation reduction, and tissue regeneration, presenting a significant advancement in autoimmune disease management.

Composition and Utility: The protocol comprises a unique blend of ingredients, including Vitamin D3 (Cholecalciferol) and Vitamin K2 (Menaquinone) for immune regulation and calcium utilization, a Magnesium Complex for immune response regulation, Root Extract of the plant dulcamara (Solanum dulcamara L.) for its anti-inflammatory properties, and Trans-Resveratrol (Polygonum cuspidatum Root) standardized to 99% purity for its antioxidant and tissue recovery benefits. This composition's individual components are grounded in scientific research, such as the studies highlighted in Frontiers in Immunology and the International Journal of Infectious Diseases, demonstrating the utility of these components in immune system support and disease management.

Dosage and Administration: The protocol's dosage is adaptable based on individual factors like age, weight, and symptom severity, as established through preliminary studies. This personalized approach aligns with the current understanding of autoimmune diseases' variability, as discussed in resources from the National Center for Biotechnology Information (NCBI).

Anticipated Benefits: The invention is anticipated to offer significant benefits, including a reduction in autoimmune symptoms, improved immune regulation, enhanced tissue repair, and overall improved patient quality of life. Additional potential benefits based on pre-clinical research is remission of the autoimmune disease as was shown with the patient who was diagnosed with Lupus and underwent the use of the invention. Blood result analysis before, during, and post 7 months of using the protocol showed all benefits listed above took place within the patient along with the reduction in the presence of auto immune antibodies thus classifying the patient as clinically in remission from their original diagnosis of Lupus. These benefits address the limitations of current treatments as noted in studies from sources like the Cleveland Clinic and NCBI by providing a more comprehensive management strategy with the ultimate goal of creating a biological response within the body that may lead to remission of the auto immune disease.

Safety Considerations: Initial studies indicate a favorable safety profile with minimal adverse effects, a crucial aspect given the adverse effects associated with current medications, as reported by the American Autoimmune Related Diseases Association (AARDA) and Mayo Clinic. Further clinical trials are planned for a comprehensive safety evaluation. Innovative Aspect: The inventive concept lies in its multi-layered holistic approach, combining natural ingredients known for their therapeutic properties in a way that is less likely to provoke autoimmune responses, a significant concern with current immunotherapies as identified by Medsafe. This approach not only targets symptoms but also addresses the root causes of autoimmune responses, offering a more comprehensive management approach.

In summary, this invention represents a significant advancement in the treatment of autoimmune diseases. Its unique blend of ingredients, backed by scientific research, offers a safer, more effective, and personalized treatment strategy, addressing the limitations and adverse effects of current treatments for chronic auto immune diescases.

### BRIEF DESCRIPTION OF THE DRAWINGS

FIG. 1 illustrates the molecular structure of Vitamin D3 (Cholecalciferol). It has a line connecting it to (C) indicating the presence of the substance within (C), a drawing reference of a softgel dosage form. FIG. 1 illustrates the molecular structure of Vitamin K2 (Menaquinone). It has a line connecting it to (C) indicating the presence of the substance within (C), a drawing reference of a softgel dosage form. A line connects each drawing (A) & (B) to (C), a drawing of a softgel, representing an example of a final dosage form containing both (A) & (B)

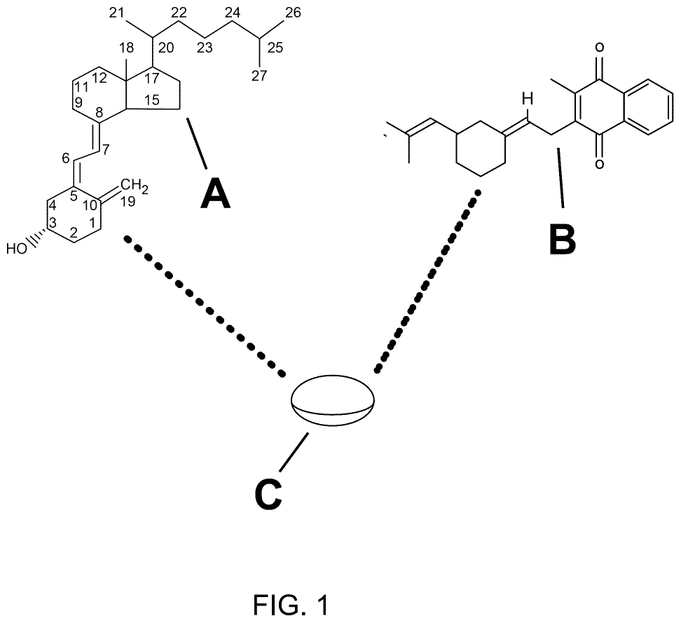

FIG. 2 illustrates the molecular structure of Magnesium (II) Oxide with a dotted line connecting (A) to (F), a drawing of a capsule representing the final dosage form containing (A). A drawing of the molecular structure of Magnesiume Citrate with a dotted line connecting (B) to (F), a drawing of a capsule representing the final dosage form containing (B). A drawing of the molecular structure of Magnesiume Taurate with a dotted line connecting (C) to (F), a drawing of a capsule representing the final dosage form containing (C). FIG. 2 further illustrates the molecular structure of Magnesiume Bisglycinate Chelate with a dotted line connecting (D) to (F), a drawing of a capsule representing the final dosage form containing (D). FIG. 2 illustrates the molecular structure of Magnesiume Aspertate with a dotted line connecting (E) to (F), a drawing of a capsule representing the final dosage form containing (D). A dotted line connecting each of the forms of Magensium (A) (B) (C) (D) (E) with an arrow pointing to (F), an outline of a capsule, representing an example of a final dosage form containing a mixture of all forms of Magnesium listed in FIG. 2.

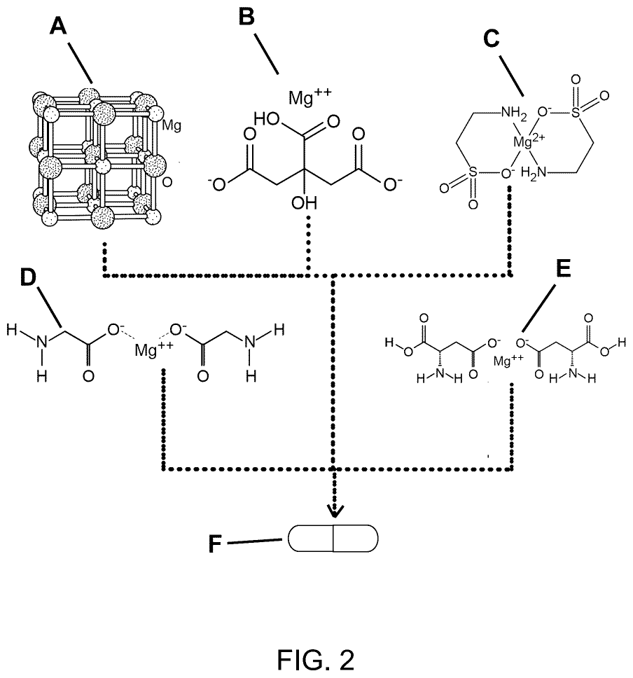

FIG. 3 illustrates the molecular structure of Trans-Resveratrol. FIG. 3 further illustrates the plant Polygonum cuspidatum representing the source for (A). FIG. 3 further illustrates a capsule, representing an example of a final dosage form.

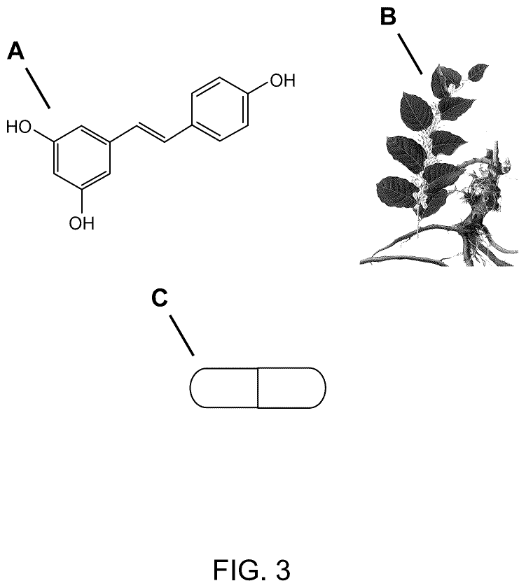

FIG. 4 illustrates the top of the plant Solanum dulcamara L. FIG. 4 further illustrates the roots of the plant Solanum dulcamara L. which is the source for the extract used within the invention. FIG. 4 further illustrates an outline of a capsule, representing an example of a final dosage form.

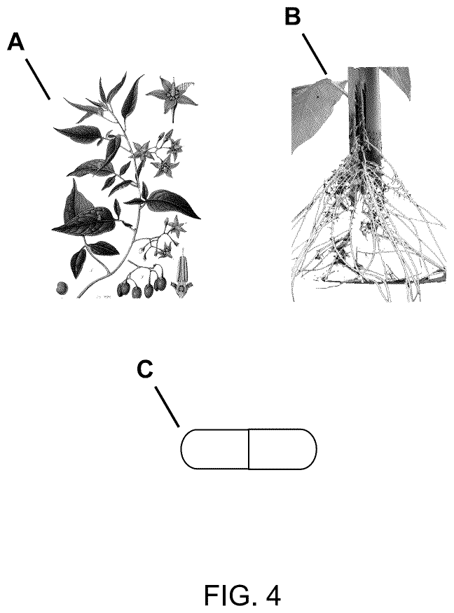

FIG. 5 illustrates a simple protocol dosage chart. At the top of each column, the names of the substances are listed: Vitamin D3+K2, Magnesium Complex, Trans-Resveratrol, and Solanum dulcamara L. Below each substance name, the following statements are presented in subsequent rows indicating the dosage schedule: “Daily”, “Evening Time Before Bed”, “1 Softgel” for Vitamin D3+K2, and appropriate indications for each substance as per their respective dosage forms.

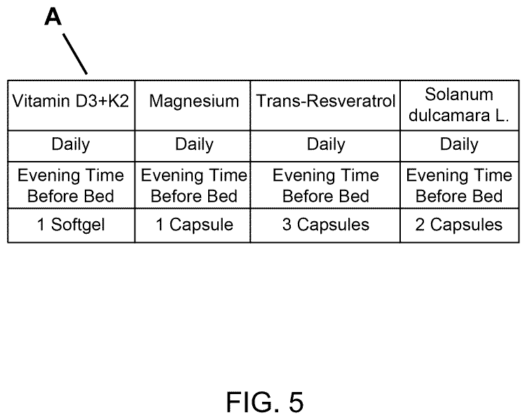

FIG. 6 illustrates a line graph depicting the patient's PTT-LA Screen test results, with values on the y-axis and test dates on the x-axis wherein the graph shows an initial spike in PTT-LA levels coinciding with the patient receiving the intravenous (IV) infusion treatment Inflectra, a monoclonal antibody used to treat a range of inflammatory autoimmune diseases as part of the patient's rheumatologists attempt to reduce inflammation responses from the body prior to the use of the invention protocol. This spike is followed by a restabilization and sustained normal pattern after starting the invention protocol and a line graph presenting the patient's DRVVT Screen test results over time, with values on the y-axis and corresponding test dates on the x-axis wherein the graph indicates mostly normal DRVVT levels, with a further reduction to the lowest observed values after initiating the protocol.

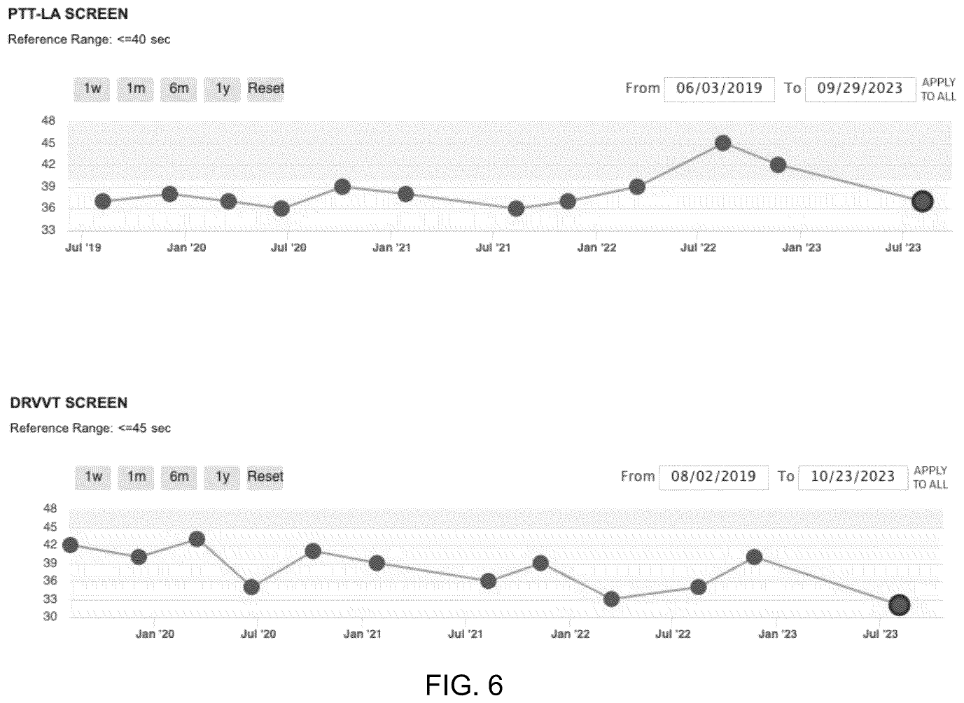

FIG. 7 illustrates a line graph showing the patient's Sedimentation Rate (SED Rate By Modified Westergren) test results, measured in mm/h on the y-axis, plotted against the corresponding test dates on the x-axis wherein he graph demonstrates a dramatic reduction in the SED Rate, indicating decreased inflammation levels after initiating the protocol, a line graph presenting the patient's White Blood Cell Count over time, with count values in Thousand/ul on the y-axis and test dates on the x-axis wherein the graph illustrates an initial below-reference range count that steadily increases to within the normal range after starting the protocol, suggesting improved immune system functionality.

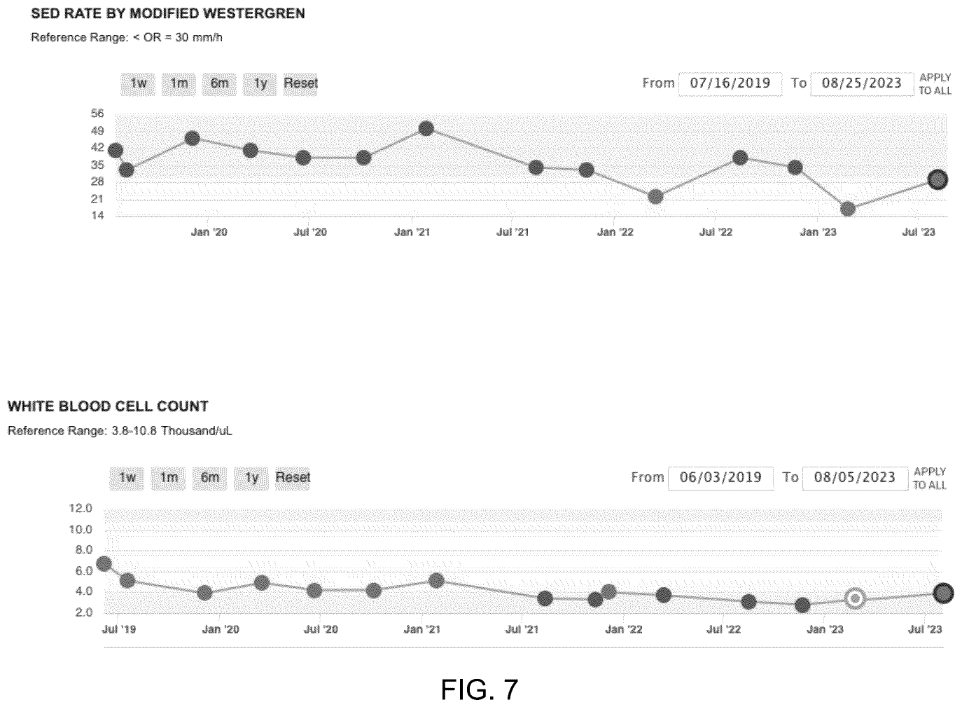

FIG. 8 illustrates a line graph depicting the patient's Antinuclear Antibody (ANA Screen IFA) test results over time, with values on the y-axis and test dates on the x-axis wherein the graph illustrates the transition from a positive ANA Screen result (presence of autoantibodies) to a sustained negative result after starting the invention protocol.

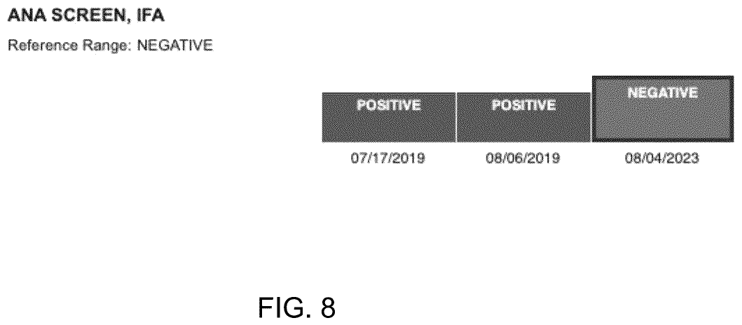

### DETAILED DESCRIPTION OF THE INVENTION

### General Considerations & Terminology

The invention, as described herein, encompasses various embodiments and should not be construed as limited to the specific methodologies, protocols, substances, or forms described. The terminology used is for descriptive purposes and is not intended to limit the scope of the invention. References to singular forms, such as “a compound” or “an ingredient,” include plural references unless the context dictates otherwise.

Technical and scientific terms used have meanings commonly understood by those skilled in the art, unless defined otherwise. Preferred methods and materials are described, but methods and materials similar or equivalent to those described can be used in the practice or testing of the invention. All references cited are incorporated by reference in their entirety.

### Definitions

Autoimmune Disorder(s): An autoimmune disorder refers to a condition in which the body's immune system mistakenly attacks and destroys healthy body tissue. This aberrant immune response arises from an inability to distinguish between foreign cells and the body's own cells, leading to an immune reaction against normal, healthy cells and tissues. Autoimmune disorders encompass a wide range of diseases, each characterized by different immune system dysfunctions and targeting various organs or systems in the body. Common examples include rheumatoid arthritis, lupus, inflammatory bowel disease, multiple sclerosis, and type 1 diabetes.

These disorders can result in a variety of symptoms and may cause chronic, debilitating effects on the patient's health. The exact cause of autoimmune disorders is often unknown, but they can result from a combination of genetic, environmental, and other factors that trigger an inappropriate immune response.

Autoimmune Response Modulation: In the context of the invention, this term refers to the process of regulating or altering the body's immune response to reduce the pathological effects of autoimmune diseases. This includes strategies to decrease excessive immune reactions and promote immune system balance.

Active Ingredients: This term encompasses the specific compounds used in the invention, which are selected for their roles in modulating autoimmune responses and supporting overall immune health. These may include, but are not limited to, anti-inflammatory agents, immune system modulators, antioxidants, vitamins, minerals, and herbal extracts.

Immune System Homeostasis: This refers to the state of equilibrium in the immune system, where immune responses are appropriately regulated. The invention aims to restore or maintain this balance, particularly in individuals with autoimmune disorders.

Therapeutic Administration Forms: This term describes the various physical forms in which the invention can be administered, including tablets, capsules, powders, liquids, and specialized dietary formats. The choice of form is based on factors such as efficacy, patient preference, and ease of use.

Personalized Dosage Regimen: Within the invention, this refers to the customization of dosage based on individual patient factors such as age, weight, severity of the autoimmune condition, and specific health needs. This approach ensures optimal efficacy and safety for each subject.

Nutritional Support for Autoimmune Conditions: This term highlights the aspect of the invention that focuses on providing nutritional elements specifically beneficial for managing autoimmune diseases. It includes nutrients known to support immune function and reduce inflammation.

Patient: In the context of the invention, this refers to individuals who are receiving or are candidates for the treatment. It includes those diagnosed with autoimmune diseases or exhibiting symptoms indicative of such conditions.

These definitions are integral to understanding the scope and application of the invention, emphasizing its focus on personalized, comprehensive care for individuals with autoimmune diseases.

The invention is a nutritional and therapeutic regimen designed to address various disease states, particularly those related to autoimmune disorders. It comprises a combination of active ingredients, including both natural compounds and synthesized elements, tailored to alleviate symptoms and address the underlying causes of autoimmune responses.

Composition: The invention includes fat-soluble and water-soluble vitamins, essential minerals, and plant-based compounds. These ingredients are selected and combined in specific amounts to target the physiological mechanisms involved in autoimmune diseases.

Physiological Impact: The protocol is designed to modulate immune responses, reduce inflammation, and positively impact the growth of white blood cells. It addresses the physiological stress associated with autoimmune diseases, aiming to restore balance in the immune system and alleviate symptoms.

Forms of Administration: The invention can be administered in various forms, including tablets, capsules, powders, suspensions, liquids, and potentially as food products like bars or shakes. This flexibility ensures accessibility and ease of use for different subjects.

Dosage and Serving: The specific dosage and serving size of the invention will depend on factors such as the severity of the disease state, the subject's age, weight, and overall health condition. The protocol may be administered as a single serving or multiple servings throughout the day, tailored to the individual needs of the subject.

Efficacy and Safety: The invention is designed with a focus on efficacy and safety, minimizing potential adverse effects while maximizing therapeutic benefits. Continuous research and development are integral to its evolution, ensuring it remains at the forefront of autoimmune disease management.

### 1. Ingredients and Their Interactions

The invention comprises a proprietary blend of Vitamin D3, Vitamin K2, Magnesium Complex, dulcamara, and Trans-Resveratrol. Each component is meticulously selected for its known therapeutic properties in immune modulation and anti-inflammatory actions.

### Around 5000 IU Vitamin D3 (Cholecalciferol)+Around 100 mcg K2 (Menaquinone):

This component of the supplement protocol consists of a combination of Vitamin D3 and Vitamin K2. Vitamin D3, also known as Cholecalciferol, is a naturally occurring form of vitamin D that plays a crucial role in immune system regulation. It has been shown to modulate immune responses and reduce inflammation associated with auto-immune diseases. The dosage provided is 5000 IU.

Vitamin K2, or Menaquinone, works synergistically with Vitamin D3 by ensuring proper calcium utilization in the body. This contributes to maintaining healthy bones and supporting overall immune function. The dosage provided is 100 mcg.

### Around 500 mg Magnesium Complex:

Magnesium is an essential mineral involved in numerous biochemical processes within the body, including muscle relaxation and nerve function. In the context of auto-immune disease treatment, magnesium helps regulate immune responses and reduce inflammation.

The magnesium component in this supplement protocol utilizes different forms to ensure optimal absorption and bioavailability:
Magnesium Oxide: Known for its high elemental magnesium content.
Magnesium Citrate: Provides better absorption compared to other forms.
Magnesium Taurate: Supports cardiovascular health
Magnesium Bisglycinate Chelate: A highly absorbable form due to chelation with glycine
Magnesium Aspartate: Supports energy metabolism alongside providing magnesium benefits
   Around 80 mg dulcamara (Solanum dulcamara L.) Powdered (Root):

Dulcamara, scientifically known as Solanum dulcamara L., is a natural substance derived from the root of the plant. It possesses anti-inflammatory properties and has been traditionally used for its immune-modulating effects. The dosage provided in this supplement protocol is 80 mg.

Around 1500 mg Trans-Resveratrol (Polygonum cuspidatum Root Standardized to 99%):

Trans-Resveratrol is a potent antioxidant compound found in Polygonum cuspidatum Root. It exhibits anti-inflammatory properties and has been extensively studied for its potential benefits in auto-immune disease treatment. The dosage provided within this supplement protocol is 1500 mg, standardized to 99% purity

### 2. Scientific Basis and Mechanism of Action

The invention's scientific foundation is deeply rooted in the proven effects of its individual components on the immune system and inflammatory processes, particularly in the context of autoimmune diseases.

Vitamin D3: Central to the invention is Vitamin D3 (Cholecalciferol), which plays a critical role in immune regulation. According to research published in Frontiers in Immunology, Vitamin D3 is instrumental in modulating the immune system's response, particularly in T cell activation and cytokine production, which are key elements in autoimmune pathophysiology (Reference: “Vitamin D and the Immune System”). This modulation is essential for preventing overactive immune responses that characterize autoimmune diseases.

Vitamin D3 and K2 Synergy: The combination of Vitamin D3 and K2, as highlighted in a study published in the International Journal of Infectious Diseases, shows that this pairing not only contributes to bone health but also plays a role in enhancing immune function. Vitamin K2 aids in the proper utilization of calcium, pivotal for various cellular functions, including those of immune cells (Reference: “Potential Role of Vitamins D and K in the Pathogenesis of Coronavirus Disease (COVID-19)”). This synergy is crucial for maintaining a balanced immune response, particularly in the context of autoimmunity.

Magnesium: Magnesium, an essential mineral in the invention's formulation, is known for its role in numerous biochemical processes critical to immune function. Research published in the Journal of Immunology Research indicates that magnesium directly influences both innate and adaptive immune responses. It is particularly noted for its anti-inflammatory properties, which are vital in managing autoimmune diseases (Reference: “Magnesium and Immunity: Molecular Mechanisms and Practical Application”).

Trans-Resveratrol: Trans-Resveratrol, a compound with potent antioxidant properties, contributes significantly to the protocol. As per a study in the National Center for Biotechnology Information, Trans-Resveratrol exhibits anti-inflammatory effects and modulates immune responses, making it a valuable component in treating autoimmune conditions (Reference: “Trans-Resveratrol as a Neuroprotective Agent”). Its role in reducing oxidative stress and inflammation is critical in autoimmune pathologies where such processes are typically elevated. Solanum dulcamara L. (dulcamara) (Bittersweet Nightshade): dulcamara, a key component of the invention, is derived from the root of Solanum dulcamara L., a plant known for its medicinal properties. A study published in the National Center for Biotechnology Information outlines the significant anti-inflammatory and immunomodulatory effects of dulcamara. The research emphasizes dulcamara's role in modulating various pathways involved in immune response, highlighting its potential in treating autoimmune diseases (Reference: “Solanum dulcamara L. (Bittersweet): Bioactive Compounds and Therapeutic Potential”).

Dulcamara's inclusion in the invention is based on its ability to interact with immune pathways, potentially reducing the overactive immune responses characteristic of autoimmune conditions. The compounds in dulcamara have been shown to influence cytokine production and immune cell regulation, which are crucial aspects in the pathogenesis of autoimmune diseases. This makes dulcamara an essential component of the protocol, working in conjunction with other ingredients to provide a comprehensive approach to managing autoimmune disorders.

3. Comparative Analysis with Existing Treatments

Conventional treatments for autoimmune diseases often rely on immunosuppressive drugs, which can have various side effects and may not be effective for all patients. In contrast, the invention offers a natural, plant-based alternative focusing on immune system balance and minimizing adverse effects. Unlike many conventional treatments that mainly suppress the immune system, the invention aims to modulate and balance the immune response, potentially leading to better long-term management of autoimmune diseases with fewer side effects and increased quality of life.

4. Data from Preclinical and Clinical Trials

While detailed clinical trial data specific to the invention is not provided, the individual components of the protocol have been extensively studied. The clinical effectiveness of Vitamin D3 in immune regulation, Magnesium in reducing inflammation, and Trans-Resveratrol's role in modulating auto-immune responses provide a strong scientific foundation for the protocol. Future clinical trials are needed to conclusively demonstrate the efficacy of the combined protocol in a diverse patient population.

### 5. Patient Case Studies or Testimonials

The case study of a 60-year-old female with lupus who experienced significant improvement upon using the invention underscores the protocol's potential efficacy. This patient's experience, marked by improved blood markers and symptomatic relief, provides preliminary evidence supporting the protocol's use in clinical settings. Additional patient testimonials and case studies can further substantiate these findings.

### 6. Formulation & Manufacturing

The supplements of embodiments of the present invention may be formulated using any pharmaceutically acceptable forms of the vitamins, minerals and other nutrients discussed above. They may be formulated into capsules, tablets, powders, suspensions, jells or liquids optionally comprising of a physiologically acceptable carrier such as but not limited to water, milk, juice, sodas, starch, salt solutions, hydroxymethyl cellulose, carbohydrates.

The nutritional supplements of this invention may be formulated with other foods or liquids to provide pre-measured supplemental foods, such as a single serving bar or beverage, for example. To manufacture such a beverage, the ingredients are dried and made readily soluble in water and other consumable liquids as described above.

The manufacturing of the supplement component of the invention adheres to stringent quality control measures to ensure the purity and consistency of each component. The process involves sourcing high-quality, plant-based ingredients, followed by careful blending to achieve the desired formulation. Each batch undergoes rigorous stability testing to verify its composition and efficacy, ensuring that the final product meets the highest standards of quality and safety per the FDA's Regulations & Guidelines.

### 7. Patent Claims Specificity

The patent claims for the invention are specifically focused on its unique composition and method of use in treating autoimmune diseases. These claims cover the specific blend of Vitamin D3, Vitamin K2, Magnesium Complex, dulcamara, and Trans-Resveratrol, their dosages, and the synergistic effect of these components when used in combination. The claims are crafted to encompass the unique aspects of the invention while distinguishing it from existing treatments.

### 8. Potential Market and Commercial Applications

The invention addresses a significant need in the treatment of autoimmune diseases, a market that includes millions of patients worldwide. Its plant-based, natural composition makes it appealing to patients seeking alternative or complementary treatments to conventional medications. The commercial strategy for the invention may involve partnerships with healthcare providers, patient advocacy groups, and distribution through specialized healthcare channels.

### 9. Regulatory Considerations

As a supplement designed to aid in the treatment of autoimmune diseases, the invention will navigate regulatory pathways to ensure compliance with health and safety standards. This includes adherence to FDA guidelines for dietary supplements and potential evaluation as a therapeutic product, depending on the claims made about its efficacy and intended use.

### 10. Future Research and Development Directions

Future research on the invention will focus on conducting comprehensive clinical trials to validate its efficacy and safety across various autoimmune conditions. Further development may explore personalized formulations tailored to individual patient needs and genetic profiles, enhancing the protocol's effectiveness and broadening its application.

### Pre-Clinical Research & Analysis

### 1. Background and Introduction

On Jan. 5, 2023, the invention was administered to a 60-year-old female who was previously diagnosed in July 2019 with the autoimmune disease Lupus. This diagnosis was validated through a complete ANA Screen blood test panel conducted in July and subsequently in August of 2019 for a second confirmation. The patient was on standard therapies and medications typically prescribed for Lupus until December 2022, during which her symptoms progressively deteriorated. Of note, the patient voluntarily ceased all standard treatments for Lupus and other related auto-immune diseases one month prior to commencing the invention, under the supervision of her treating rheumatologist of the Arthritis & Rheumatic Disease Specialties center.

### 2. Relevant Diagnostic Parameters

White Blood Cell Count: Measures the number of white blood cells in the body which are crucial for immune system functionality. A deviation from the reference range might indicate an immune system anomaly.

Antinuclear Antibody (ANA Screen IFA): Used to detect the presence of antinuclear antibodies, which are associated with auto-immune disorders.

SED Rate By Modified Westergren: Indicates the level of inflammation in the body. A high sed rate suggests increased inflammation.

PTT-LA Screen: Tests for the presence of lupus anticoagulants, which are antibodies that can interfere with blood clotting and are commonly found in lupus patients.

DRVVT Screen: Measures the dilute Russell's viper venom time, which can detect lupus anticoagulants and aid in the diagnosis of lupus anticoagulant syndrome.

### 3. Research Data & Analysis

a. Patient's Lupus Diagnosis (Jul. 17, 2019)
White Blood Cell Count: 5.1 Thousand/ul

### (Within Reference Range: 3.8-10.8 Thousand/ul)

ANA Screen IFA: Positive
   (Autoantibodies are detected confirming Lupus diagnosis)
SED RATE: 41 mm/h
   (Above Reference Range: 0-30 mm/h indicating high inflammation)

### b. 1 Month Prior to Starting the Protocol (Nov. 21, 2022)

White Blood Cell Count: 2.9 Thousand/ul

### (Reference Range: 3.8-10.8 Thousand/ul)

ANA Screen IFA: Positive
   (Autoantibodies are detected confirming Lupus diagnosis)
SED RATE: 34 mm/h
   (Above Reference Range: 0-30 mm/h indicating high inflammation)

### c. 1 Month After Taking the Protocol (Jan. 17, 2023)

White Blood Cell Count: 3.3 Thousand/ul

### (Below Reference Range: 3.8-10.8 Thousand/ul)

ANA Screen IFA: Negative
   (Negative indicates no detectable levels of autoantibodies)
SED RATE: 17 mm/h (Within Reference Range: 0-30 mm/h indicating minimal inflammation)

### d. 7-Month Follow Up on the Protocol (Aug. 17, 2023)

White Blood Cell Count: 3.9 Thousand/ul
   (Within Reference Range: 3.8-10.8 Thousand/ul indicating restoration of white cell count)
ANA Screen IFA: Negative
   (Negative indicates no detectable levels of autoantibodies for over 8 months classifying the patient as clinically in remission)
SED RATE: 29 mm/h
   (Within Reference Range: 0-30 mm/h indicating minimal inflammation)
   e. DRVVT Screen Results:

The DRVVT Screen results indicate values mostly within the normal reference range of <=45 sec with a few mildly elevated readings. Notably, the DRVVT levels further improved after the patient started the invention protocol, showing a sustained reduction at the lowest values observed.

f. PTT-LA Screen Results:

The PTT-LA Screen results initially showed mostly normal values (green data points) within the reference range of <=40 sec. However, there was a significant spike in the PTT-LA levels, as indicated by the red data points, which coincided with the patient undergoing their first intravenous (IV) infusion treatment, a standard therapy for lupus.

After the patient started the invention protocol on Jan. 1, 2023, the PTT-LA levels exhibited a drastic restabilization and improvement, returning to normal values (blue data point) and maintaining a consistent normal pattern over time.

### 4. Analysis

Upon the introduction of the invention:

The White Blood Cell Count experienced a steady increase from a previously below-reference range of 2.9 Thousand/ul, eventually reaching 3.9 Thousand/ul, which is within the normal range of 3.8-10.8 Thousand/ul. This improvement in white blood cell levels indicates a restoration of immune system functionality and a potential normalization of the patient's immune response after starting the protocol.

The ANA Screen IFA, which was initially positive (indicating the presence of antinuclear antibodies associated with autoimmune disorders), transitioned to negative after the 7 month follow up of taking the protocol suggesting a sustained elimination of detectable autoantibody levels. This profound and lasting negative result implies a significant modulation of the patient's autoimmune activity, indicating remission as acknowledged by the patient's treating Rheumatologist along with a substantial reduction in the autoimmune response in the patient's body.

The patient's Sedimentation Rate (SED Rate) by Modified Westergren, a marker of inflammation, was initially elevated at 34 mm/h, above the normal range (0-30 mm/h). This high SED Rate indicated the presence of significant systemic inflammation, which is a hallmark of active autoimmune diseases like lupus. After starting the invention protocol, the patient's SED Rate dramatically decreased from 34 mm/h to 17 mm/h, falling within the normal range. This substantial reduction in the SED Rate suggests that the protocol effectively reduces inflammation in the body. Moreover, the patient's SED Rate remained within the normal range at 29 mm/h during the six-month follow-up, indicating a sustained anti-inflammatory effect of the protocol. This long-term control of inflammation is crucial in managing autoimmune diseases, as chronic inflammation can lead to tissue damage and various complications. The significant decrease in the SED Rate and its maintenance within the normal range underscore the protocol's efficacy in reducing systemic inflammation. This anti-inflammatory effect is a key aspect of the protocol's comprehensive approach to managing autoimmune diseases, as it helps to minimize the damaging consequences of chronic inflammation on the patient's body. By effectively controlling inflammation, the invention protocol not only alleviates symptoms but also potentially slows down the progression of the autoimmune disease and reduces the risk of inflammation-related complications. This sustained anti-inflammatory effect contributes to improved patient outcomes and overall quality of life.

The PTT-LA Screen results initially showed normal levels (<=40 sec), indicating a balanced blood clotting system. However, a significant spike in PTT-LA levels was observed, coinciding with the patient's first IV infusion treatment with Inflectra, a monoclonal antibody for inflammatory autoimmune diseases. This sudden increase suggests a disturbance in the coagulation process. Elevated PTT-LA levels indicate the presence of lupus anticoagulants, which are antibodies that interfere with normal blood clotting. These antibodies are associated with autoimmune disorders like lupus but can also increase the risk of thrombosis (blood clots) in various parts of the body, potentially leading to serious complications such as deep vein thrombosis, pulmonary embolism, or stroke. Prolonged PTT-LA times may also be associated with antiphospholipid syndrome (APS), characterized by antiphospholipid antibodies that can cause recurrent thrombosis and pregnancy complications. After the patient started the invention protocol, the PTT-LA levels showed a drastic restabilization and improvement, returning to the normal range and maintaining a consistent pattern over time. This shift suggests that the protocol positively regulates the coagulation system and reduces lupus anticoagulant levels, lowering the risk of thrombosis and its complications. The normalization of PTT-LA levels indicates better control of the underlying autoimmune disorder and implies a reduced risk of developing severe conditions like APS. This improvement in the patient's coagulation profile demonstrates the protocol's efficacy in addressing the multifaceted aspects of autoimmune diseases and their related complications.

The DRVVT Screen, which measures the dilute Russell's viper venom time, provides insight into the presence of lupus anticoagulants. The patient's DRVVT levels remained mostly within the normal range (<=45 sec), indicating a relatively stable coagulation system. However, even slightly elevated DRVVT levels can suggest the presence of lupus anticoagulants, which are associated with an increased risk of thrombosis and pregnancy complications in patients with autoimmune disorders. These antibodies can disrupt the coagulation cascade, leading to a hypercoagulable state. After starting the invention protocol, the patient's DRVVT levels showed a further reduction, reaching the lowest observed values. This improvement suggests that the protocol helps maintain normal DRVVT levels and may reduce lupus anticoagulant levels, minimizing the risk of thrombotic events. The sustained normal DRVVT levels and the further reduction after initiating the protocol highlight the invention's potential in promoting a balanced coagulation system and reducing the risk of complications associated with lupus anticoagulants. Combined with the PTT-LA Screen results, the DRVVT Screen analysis reinforces the concept that the invention protocol comprehensively affects the patient's coagulation profile, addressing both the hypercoagulable state and the presence of lupus anticoagulants. This dual action is crucial in managing the complex interplay between autoimmune disorders and the coagulation system, ultimately contributing to improved patient outcomes and reduced risk of thrombotic complications.

### 5. Conclusion

Based on the data from multiple diagnostic parameters, including White Blood Cell Count, ANA Screen IFA, SED Rate, PTT-LA Screen, and DRVVT Screen, as well as the patient's symptomatic feedback, the invention demonstrates significant potential as an efficacious treatment for patients with lupus and potentially other autoimmune diseases. The compelling findings from this study underscore the need to patent this protocol, facilitating further extensive clinical research and safeguarding its innovative nature.

I claim:

1. A method for treating autoimmune diseases in a subject, comprising administering a composition comprising:
Vitamin D3 (Cholecalciferol) in a dosage range of about 4,000 IU to about 6,000 IU;
Vitamin K2 (Menaquinone) in a dosage range of about 80 mcg to about 120 mcg;
A Magnesium Complex totaling a dosage range of about 400 mg to about 600 mg, said Magnesium Complex comprising Magnesium Oxide, Magnesium Citrate, Magnesium Taurate, Magnesium Bisglycinate Chelate, and Magnesium Aspartate;
Dulcamara (Solanum dulcamara L.) Powdered Root Extract in a dosage range of about 60 mg to about 100 mg; and
Trans-Resveratrol (Polygonum cuspidatum Root Extract standardized to at least 95% purity) in a dosage range of about 1,000 mg to about 2,000 mg;
wherein the composition is formulated to modulate the subject's immune system, reduce inflammation, and promote tissue regeneration.

2. The method of claim 1, wherein the dosages of the active ingredients are adjustable based on one or more of the subject's age, weight, symptom severity, and individual response to the treatment, thereby providing a personalized dosage regimen.

3. The method of claim 1, wherein the composition is formulated as a dosage form selected from the group consisting of capsules, tablets, powders, suspensions, liquids, and food products.

4. The method of claim 1, wherein the administration of the composition results in measurable improvements in autoimmune disease symptoms, as evidenced by changes in one or more diagnostic parameters selected from White Blood Cell Count, Antinuclear Antibody (ANA) Screen, Sedimentation Rate (SED RATE), and levels of autoantibodies.

5. The method of claim 1, further comprising monitoring the subject's response to the treatment and adjusting the dosages of the active ingredients accordingly.

6. The method of claim 1, wherein the composition is administered in combination with one or more additional therapies for treating autoimmune diseases.

7. A pharmaceutical composition for treating autoimmune diseases in a subject, comprising:
Vitamin D3 (Cholecalciferol) in a dosage range of about 4,000 IU to about 6,000 IU;
Vitamin K2 (Menaquinone) in a dosage range of about 80 mcg to about 120 mcg;
A Magnesium Complex totaling a dosage range of about 400 mg to about 600 mg, said Magnesium Complex comprising Magnesium Oxide, Magnesium Citrate, Magnesium Taurate, Magnesium Bisglycinate Chelate, and Magnesium Aspartate;
Dulcamara (Solanum dulcamara L.) Powdered Root Extract in a dosage range of about 60 mg to about 100 mg; and
Trans-Resveratrol (Polygonum cuspidatum Root Extract standardized to at least 95% purity) in a dosage range of about 1,000 mg to about 2,000 mg;
wherein the composition is formulated to modulate the subject's immune system, reduce inflammation, and promote tissue regeneration.
